(19) United States
(12) Patent Application Publication
(10) Pub. No.: US 2026/0176541 A1
(43) Pub. Date: Jun. 25, 2026

(54) SYSTEM AND METHOD FOR PRODUCING A FUEL PRODUCT

(71) Applicants: Compressed Energy Systems, LLC, Eldorado, IL (US); M&K Gas Company, LLC, Holdrege, NE (US)

(72) Inventor: Christopher E. SCHIMP, Eldorado, IL (US)

(21) Appl. No.: 19/259,588

(22) Filed: Jul. 3, 2025

Related U.S. Application Data
(63) Continuation-in-part of application No. 17/671,792, filed on Feb. 15, 2022, now abandoned.

(51) Int. Cl. C10L 3/08; B03B 5/66; C12P 5/02

(52) U.S. Cl. CPC C10L 3/08; B03B 5/66; C12P 5/023; C10L 2290/24; C10L 2290/26; C10L 2290/28; C10L 2290/54

(57) ABSTRACT

This disclosure relates to systems and methods for producing a fuel product from a source of organic matter which includes manure and may include other byproducts. The organic matter is processed by a teeter water feed hindered-bed separator device for substantially separating the animal manure from the other byproducts. The separated animal manure is then subjected to anaerobic digesting to produce the fuel product.

### CROSS-REFERENCES TO RELATED APPLICATIONS

This application claims priority as a continuation-in-part of prior filed U.S. application Ser. No. 17/671,792, filed Feb. 15, 2022.

### TECHNICAL FIELD

The present disclosure is generally directed to the production of a fuel product, in particular systems and methods for processing livestock waste to obtain fuel and more particularly, systems and methods for creating fuel using anaerobic digesting.

### BACKGROUND

Many livestock farms, such as dairy farms and cattle ranches produce tremendous amounts of manure waste that must be properly managed. One form of management is to use manure waste to produce a source of fuel such as a methane biogas that can be converted to electricity or otherwise used as a source of fuel similar to natural gas.

In general, the invention described herein is a system and method for recovering manure in a livestock operation, such as a dairy or cattle operation. The manure may be recovered for purposes of supplying a anaerobic digester for extraction of energy from the biodegradable material.

Anaerobic digestion refers to a collection of processes by which organic matter is broken down by microorganisms in the absence of oxygen.

Anaerobic digestion is used as part of a treatment process in the recycling of biodegradable waste matter such as food waste, sewage sludge, or animal waste. The process results in the production of biogas, which is considered a source of renewal energy. Biogas can be further refined to produce bio methane, which has a similar methane content to natural gas. There are several problems in anaerobic digestion of waste from beef cattle, dairy cows or swine. Typically, the waste from such animals includes amounts of dirt from the feed lots or sand and other materials used in bedding for the livestock. The dirt, sand and other materials cause issues with the digesters such as clogging, which then requires significant down time to clean out. In addition, typical digester systems are limited to a maximum of about ten percent total solids before the introduced slurry becomes too thick for effective anaerobic digestion. Agricultural manure is on the average about 57% volatile solids, which is the portion that generates methane in the digester. To date, there is no economical technology to remove the dirt, sand or other materials from the manure before it enters the anaerobic digesting process. This need is met by the systems and methods described herein, which feature the use of animal waste to form a fuel product.

### BRIEF DESCRIPTION OF DRAWINGS

To facilitate further description of the embodiments, the following drawings are provided, in which like references are intended to refer to like or corresponding parts, and in which:

FIG. 1 is a schematic view of a system for producing a fuel product from a source of organic matter;

FIG. 2 is a side view of an embodiment of a teeter water feed hindered-bed separator device that may be used as part of the system for producing a fuel product from a source of organic matter; and

FIG. 3 graphically represents a method for producing a fuel product from a source of organic matter.

For simplicity and clarity of illustration, the drawing figures illustrate the general manner of construction, and descriptions and details of well-known features and techniques may be omitted to avoid unnecessarily obscuring the present disclosure. Additionally, elements in the drawing figures are not necessarily drawn to scale. For example, the dimensions of some of the elements in the figures may be exaggerated relative to other elements to help improve understanding of embodiments of the present disclosure. The same reference numerals in different figures denote the same elements.

The terms “include,” and “have,” and any variations thereof, are intended to cover a non-exclusive inclusion, such that a process, method, system, article, device, or apparatus that comprises a list of elements is not necessarily limited to those elements, but may include other elements not expressly listed or inherent to such process, method, system, article, device, or apparatus.

The terms “top,” “bottom,” “upper,” “lower,” and the like in the description and in the claims, if any, are used for descriptive purposes and not necessarily for describing permanent relative positions. It is to be understood that the terms so used are interchangeable under appropriate circumstances such that the embodiments of the system and/or methods described herein are, for example, capable of operation in other orientations than those illustrated or otherwise described herein.

### DETAILED DESCRIPTION OF EXEMPLARY EMBODIMENTS

The present disclosure relates to a system and method for processing organic matter containing manure and other byproducts such as dirt, sand and other materials as part of a biogas digesting system in order to produce a fuel product. The method and system substantially separates and removes the manure from other byproducts or materials based on weight or density. The method and system uses the separated solids for feeding a digester that may be used to produce a fuel product such as a methane biogas.

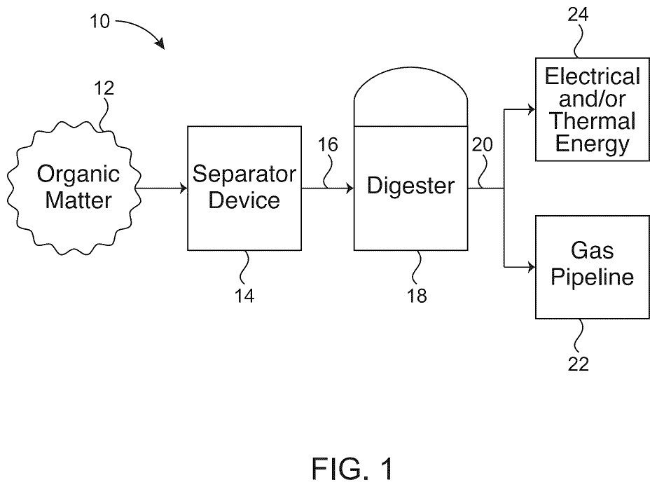

FIG. 1 is a schematic view of a system 10 for producing a fuel product from a source of organic matter 12, which may comprise animal manure and other byproducts such as dirt, sand and other materials. The organic matter 12 may be collected from the waste of livestock, such as beef cattle or dairy cows. The other materials or byproducts may be included in the animal waste due to the animals being housed in or fed in facilities or areas containing dirt or bedding that may include sand or other materials.

The organic matter 12 is fed into a separation device 14 such as a teeter water feed hindered-bed separator device. The separator device 14 substantially separates the animal manure 16 from the other materials and byproducts contained in the organic matter 12. The separated animal manure 16 may then be subjected to an anaerobic digester 18 to produce a biogas fuel product 20, which may then be processed as necessary for feeding into a natural gas pipeline 22 or used to generate electrical and/or thermal energy 24, among other uses.

Preferably, prior to feeding the organic matter 12 into the separation device 14, the organic matter 12 is prepared so that the separation device 14 can distinguish the volatile solids from the nonvolatile solids contained within the organic matter 12, which may have density differences of about 1.0 to 1.6 kg/m³. For example, the organic matter 12 may be mixed with water and fed through a macerating device to macerate or grind down the particle size of the solids to about 0.5 mm to about 3.0 mm. This particle size range includes an optimal surface area for bacteria activity for the anaerobic digestion in the digester. This particle size range is also ideal to maintain the volatile and nonvolatile solids in suspension with moderate mixing action in the digester as violent mixing generally hinders bacteria activity. Finally, this particle size range allows more efficient density separation in the teeter zone of the separator device 14.

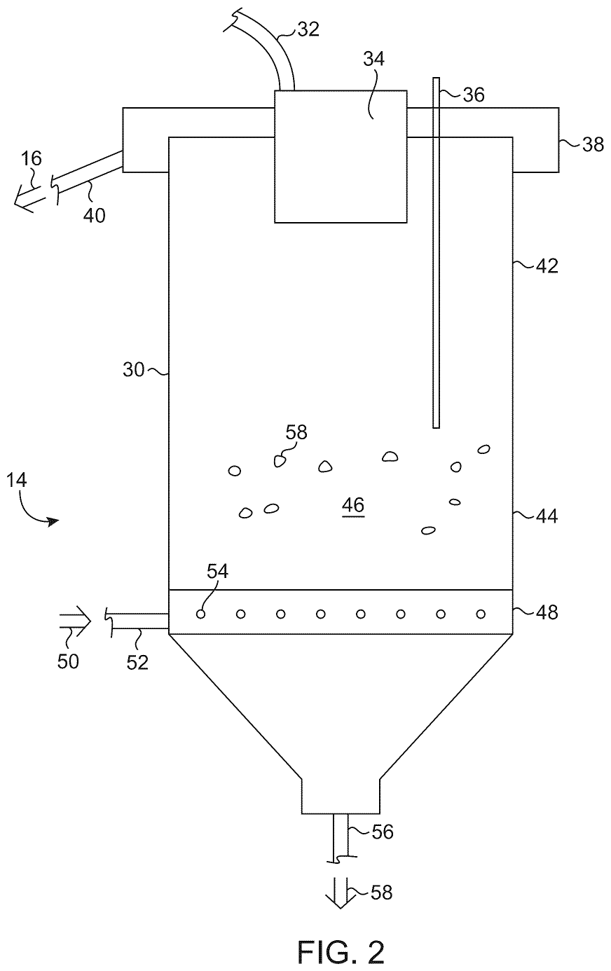

FIG. 2 depicts an embodiment of a teeter water feed hindered-bed separator device 14 that may be used as part of the system for producing a fuel product from a source of organic matter 12 consisting of animal manure and other byproducts. Although FIG. 2 depicts one form of a teeter water feed hindered-bed separator device, the present invention is not limited to any particular form of separator device.

As is known, a hindered-bed separator is a vessel in which water is evenly introduced across the base of the device and rises upward. The separator device 14 consists of a main housing 30 forming an enclosed chamber that generally includes an upper portion 42 and a lower portion 44. A cylindrical feed well 34 is positioned centrally within the upper portion 42 and extends below an overflow launder 38. The feed well 34 is connected to an inlet feed line 32 into which the slurry including organic matter 12 is controllably fed.

The separator device 14 further includes an intake pipe 52 for receiving a source of teeter water inflow 50. The intake pipe 52 is connected to a series of water injection pipes to supply pressurized teeter water through a plurality of injection nozzles 54 preferably located evenly across a teeter plate 48. In such a manner, the teeter water 50 flows upwardly toward the feed well 34.

The slurry including the organic matter 12 is introduced in the inlet 32 located in the upper end of the separator device 14 into the feed well 34. Teeter water 50 controllably enters the separator device through the intake pipe 52 and is evenly dispersed across the teeter plate 48 for dispersion through the injection nozzles 54.

The upward current of teeter water 50 creates a teeter zone 46 that suspends particles of a certain weight or density. Larger or more dense particles begin to settle downward against the flow of rising teeter water 50 at a rate defined by the particle size and/or density (specific gravity). The courser or denser solids settle at a rate that exceeds that of the rising water. These courser solids 58 may be discharged through the discharge outlet valve 56, which may be controlled by an electronic actuator that may be under computer control. The finer particles are maintained by the water flow in the upper portion 42 of the separator device 14 and discharged over the top and into the collection launder 38.

Preferably, the upward current of teeter water 50 is fresh water and generally this fresh water will mix in with and dilute the slurry including organic matter 12. The goal is to maximize the amount of volatile solids in the digester to about 10%. Thus, preferably, the slurry including organic matter 12 introduced into the digester has a maximum amount of volatile solids concentration of about 12% to about 15%.

In addition to the 10% maximum solids for the slurry mixture going into digesters for methane production, there exists another parameter for optimum methane production. By removing the nonvolatile solids with the separator the slurry mixture can then be concentrated to above the minimum of 12,000 mg/L of volatile solids. Should the concentration drop below 12,000 mg/L the bacteria may begin to cannibalize causing a decline in methane production. If the volatile solids concentration is too high the bacteria may not work properly causing a decline in methane production.

Therefore, the amount of feed has to be controlled going into the digesters for optimal methane production. The Teeter bed Separator allows this process to be controlled with precision as an agricultural nutritionist does for cattle feed and amount.

Rising water and light solids flow over the upper portion 42 of the separator device 14 and collect in an overflow launder 34. The light solids, which preferably consist of usable manure 16, may be discharged from separator device via outlet pipe 40.

The separator may also include a density control device 36 to measure the relative densities of the slurry in the upper and lower portions. If the measured density is greater than the desired density, the discharge outlet valve 56 may be regulated to adjust the average density of the teeter bed zone to a preselected standard value. This value may be obtained from empirical testing and is stored in the density control device 36 as a set point to open the discharge outlet valve 56 to dump out the nonvolatile solids. If the ratio of volatile to nonvolatile solids in the agriculture manure changes, the value will need to be determined anew. This predetermined value is obtained by testing at different particle sizes with percent of nonvolatile coming out of the discharge outlet valve 56 opened by the density control device 36 and the percent of volatile solids coming out of the top of the digester via the outlet pipe 40. The predetermined value is obtained from the optimal test results with the highest percent of volatile solids exiting the outlet pipe 40, with little to no volatile solids going out of the waste discharge outlet in the bottom of the digester.

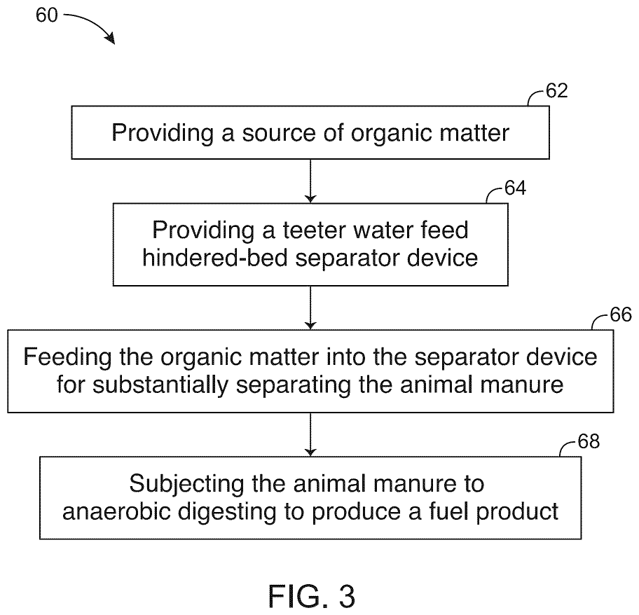

FIG. 3 is a method 60 for producing a fuel product. The method 60 begins at step 62 by providing a source of organic matter. Preferably, the organic matter includes livestock waste including animal manure, which may be contaminated with dirt, sand and other materials. The organic matter may be macerated to macerate or grind down the particle size of the solids to about 0.5 mm to about 3.0 mm. The method continues at step 64 in which a teeter water feed hindered-bed separator device is provided. In step 66, the organic matter is fed into the separator device for substantially separating the animal manure from the other materials in the organic matter. In step 68, the separated animal manure is subjected to anaerobic digesting to produce a fuel product. The method 60 may then be repeated suing an additional source of organic matter.

As is known in the biogas digester industry, in order to make a million gallon digester economical, it takes roughly 1700 cows in which each cow produces 20 gallon/day of manure, as the standard retention time for the manure in the digesters is 30 days. Each cow produces on average 22,000 cubic feet of methane per year. Utilizing Teeter bed separator technology to remove the nonvolatile solids allows a higher concentration of volatile solids that the bacteria digest for methane production. This essentially increases the amount of methane for each cow.

While various novel features of the invention have been shown, described, and pointed out as applied to particular embodiments thereof, it should be understood that various omissions, substitutions and changes in the form and details of the systems and methods described and illustrated may be made by those skilled in the art without departing from the spirit of the invention. Amongst other things, the steps shown in the methods may be carried out in different orders in many cases, where such may be appropriate. Those skilled in the art will recognize, based on the above disclosure and an understanding therefrom of the teachings of the invention, that the particular hardware and devices that are part of the system described herein, and the general functionality provided by and incorporated therein, may vary in different embodiments of the invention. Accordingly, the particular system components are for illustrative purposes to facilitate a full and complete understanding and appreciation of the various aspects and functionality of particular embodiments of the invention, as realized in system and method embodiments thereof. Those skilled in the art will appreciate that the invention can be practiced in other than the described embodiments, which are presented for purposes of illustration and not limitation.

What is claimed is:

1. A method for producing a fuel product comprising the steps of:
providing a source of organic matter, the organic matter comprising animal manure comprised of volatile and nonvolatile solids;
mixing the organic matter with water and macerating the organic matter to generate a slurry with solids having a particle size in a range of about 0.5 mm to about 3.0 mm;
providing a teeter water feed hindered-bed separator device;
feeding the slurry into the separator device for substantially separating out the nonvolatile solids from the slurry; and
subjecting the slurry to anaerobic digesting to produce the fuel product.

2. The method of claim 1 wherein the separator device separates out the nonvolatile solids based on the specific density of the volatile and nonvolatile solids.

3. The method of claim 1 wherein the fuel product is a methane biogas.

4. The method of claim 1 wherein the other byproducts include sand and/or dirt.

5. The method of claim 1 wherein the slurry subjected to anaerobic digestion has a concentration of volatile solids of about 12% to 15% of the slurry.

6. A system for producing a fuel product comprising:
a source of organic matter, the organic matter comprising animal manure comprising volatile and nonvolatile solids;
a macerator for macerating the organic matter to generate a slurry with solids having a particle size in a range of about 0.5 mm to about 3.0 mm;
a teeter water feed hindered-bed separator device for accepting the slurry and separating out the nonvolatile solids from the slurry; and
an anaerobic digester for producing the fuel product.

7. The system of claim 6 wherein the hindered-bed separator device comprises:
a main housing having an upper portion and a lower portion;
a feed inlet for feeding organic matter into the upper portion of the separator;
an inlet feeding teeter water that flows through a dispersing teeter plate and upward toward the upper portion of the main housing creating a zone of separation between the volatile and nonvolatile solids;
an overflow launder collecting the separated volatile solids; and
an outlet pipe for discharging the volatile solids.

8. The system of claim 7 further comprising a density control device to measure the relative densities in the upper and lower portion of the main housing and wherein the zone of separation may be adjusted for the specific gravity of the nonvolatile and volatile solids.
